(19) United States
(12) Patent Application Publication
(10) Pub. No.: US 2021/0156849 A1
(43) Pub. Date: May 27, 2021

(54) ANALYTICAL METHOD, REAGENT KIT AND ANALYTIC DEVICE

(71) Applicant: Canon Medical Systems Corporation, Otawara-shi (JP)

(72) Inventor: Satoru SUGITA, Nasushiobara (JP)

(73) Assignee: Canon Medical Systems Corporation, Otawara-shi (JP)

(21) Appl. No.: 17/163,623

(22) Filed: Feb. 1, 2021

Related U.S. Application Data
(63) Continuation of application No. PCT/JP2019/030103, filed on Jul. 31, 2019.

(30) Foreign Application Priority Data
Aug. 2, 2018 (JP) 2018-146102

(51) Int. Cl. G01N 33/542; G01N 21/64

(52) U.S. Cl. CPC G01N 33/542; G01N 2021/6439; G01N 21/6428

(57) ABSTRACT

According to one embodiment, an analytical method of detecting a target substance in a sample, includes mixing a) a first substance containing a stimuli-sensitive macromolecule and an environment-responsive fluorescent substance, b) a second substance containing a first capturing body, and c) a third substance containing a second capturing body labeled with an aggregation inhibitor which inhibits aggregation of the stimuli-sensitive macromolecule, with the sample, maintaining the mixture under such a condition that the stimuli-sensitive macromolecule aggregates, detecting fluorescence from the environment-responsive fluorescent substance, and determining presence/absence or quantity of the target substance in the sample.

### CROSS-REFERENCE TO RELATED APPLICATIONS

This application is a continuation Application of PCT Application No. PCT/JP2019/030103, filed Jul. 31, 2019 and based upon and claiming the benefit of priority from Japanese Patent Application No. 2018-146102, filed Aug. 2, 2018, the entire contents of all of which are incorporated herein by reference.

### FIELD

Embodiments described herein relate generally to an analytic method, a reagent kit and an analytic device.

### BACKGROUND

In recent years, methods of detecting target substances in samples using stimuli-sensitive macromolecules have been carried out. The stimuli-sensitive macromolecules refer to macromolecules which changes their polarity along with change in temperature, pH, light, salt concentration or the like.

For example, a method of detecting a target substance, which operates as follows, is reported. That is, a first affinity substance, to which temperature-responsive macromolecules are bonded, and having affinity to an object to be detected, a second affinity substance labeled with a substance having charge and having affinity to an object to be detected, and a sample are mixed together, and then subjected to a high-temperature condition to make the temperature-responsive macromolecules hydrophobic to aggregate together. Then, the aggregate is separated by magnetic force, and absorbance of the separated fraction id measured, thereby detecting the target substance.

Moreover, the ELISA method and the CLEIA method have been used as techniques of detecting a target substance in a sample at high sensitivity and in a wide range.

However, with the above-described method, it is difficult to accurately detect or quantify a target substance when the amount thereof is very small. On the other hand, the ELISA method and the CLEIA method each indispensably require separation and wash during the process, and the operation thereof is complicated. With the present embodiments, an analytical method, a reagent kit, and an analytic device which are simpler and of higher precision can be provided.

### BRIEF DESCRIPTION OF THE DRAWINGS

FIG. 1 is a schematic diagram showing examples of the first to third substances of an embodiment.

FIG. 2 is a flow chart illustrating an example of an analytical method of the embodiment.

FIG. 3 is a schematic diagram showing examples of complexes of the embodiment.

FIG. 4 is a schematic diagram showing an example of a complex and an aggregate of the embodiment.

FIG. 5 is a schematic diagram showing another example of a complex and an aggregate of the embodiment.

FIG. 6 is a plan view showing an example of an analyzing unit of an auto analyzer of the embodiment.

FIG. 7 is a block diagram showing an example of the auto analyzer of the embodiment.

FIG. 8 is a schematic diagram showing examples of the first to third substances of the embodiment.

FIG. 9 is a schematic diagram showing a process of an analytical method which uses the auto analyzer of the embodiment.

FIG. 10 is a schematic diagram showing examples of the first to third substances of the embodiment.

FIG. 11 is a flowchart illustrating an example of the analytical method of the embodiment.

FIG. 12 is a schematic diagram showing an example of the complex of the embodiment.

FIG. 13 is a schematic diagram showing examples of a complex and an aggregate of the embodiment.

FIG. 14 is a schematic diagram showing examples of the complex and aggregate of the embodiment.

### DETAILED DESCRIPTION

In general, according to one embodiment, there is provided an analytical method of detecting a target substance in a sample. The analytical method comprising: mixing, with a sample, a) a first substance containing a stimuli-sensitive macromolecule and an environment-responsive fluorescent substance bonded to one end of the stimuli-sensitive macromolecule, b) a second substance containing a first capturing body which bonds specifically to a target substance, and c) a third substance containing a second capturing body labeled with an aggregation inhibitor which inhibits aggregation of stimuli-sensitive macromolecule, which bonds specifically to the target substance; maintaining the mixture under a condition that stimuli-sensitive macromolecules aggregate; detecting fluorescence from the environment-responsive fluorescent substance; and determining presence/absence of the target substance in the sample existence or a quantity thereof based on a result of the detecting.

Analytical Method

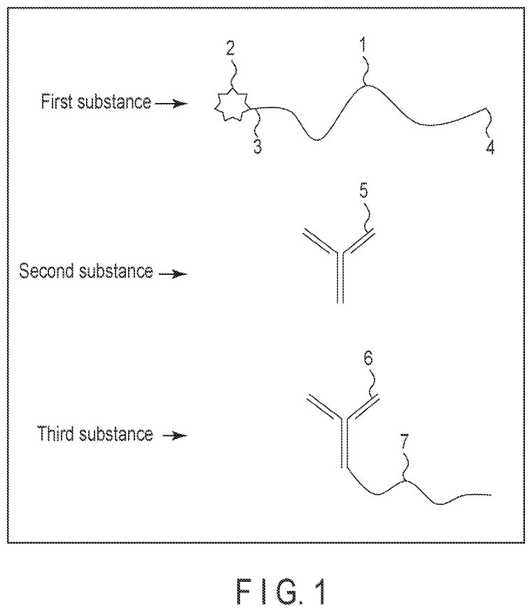

The analytical method according to an embodiment is a technique of detecting a target substance in a sample. The analytical method of the embodiment is carried out using the first to third substances. FIG. 1 is a schematic diagram showing examples of first to third substances.

The first substance contains a stimuli-sensitive macromolecule and an environment-responsive fluorescent substance.

Stimuli-sensitive macromolecules are substances whose solubility to water changes reversibly under a specific condition. That is, in an aqueous solution under a certain condition, the macromolecules are hydrophilic and therefore do not aggregate, but becomes hydrophobic under a specific condition different from the above condition, and aggregate by hydrophobic bonding. Here, in an example which will be provided, the stimuli-sensitive macromolecule is a temperature-responsive macromolecule 1. The temperature-responsive macromolecule 1 is hydrophilic under a low-temperature condition and does not aggregate, but becomes hydrophobic under a high temperature condition and aggregate by hydrophobic bonding. The stimuli-sensitive macromolecule is not limited to a temperature-responsive macromolecule, and some other type of macromolecule may be used.

Environment-responsive fluorescent substance is a fluorescent substance whose wavelength of fluorescence to be emitted changes depending on the surrounding environment. Here, in the example, the environment-responsive fluorescent substance is a polarity-responsive fluorescent substance 2. The polarity-responsive fluorescent substance 2 is a fluorescent substance whose wavelength of fluorescence to be emitted changes depending on the surrounding polarity, that is, whether hydrophilic or hydrophobic. The environment-responsive fluorescent substance is not limited to the polarity-responsive fluorescent substance, but some other type of fluorescent substance may be used.

The polarity-responsive fluorescent substance 2 is bonded to one end 3 of the temperature-responsive macromolecule 1.

The second substance contains a first capturing body 5. The first capturing body 5 is a substance which can bond specifically to a target substance. In this example, the first capturing body 5 is an antibody.

The first capturing body 5 and another end 4 of the temperature-responsive macromolecule 1 are constituted so as to bond to each other. The site of the first capturing body 5, which is to bond to the temperature-responsive macromolecule 1 does not affect bonding to a target substance.

The third substance includes a second capturing body 6 labeled with an aggregation inhibitor 7. The second capturing body 6 is a substance to specifically bond to a target substance. It is preferable that the first capturing body and the second capturing body be constituted so as to bond to different sites of the target substance, respectively. In this example, the second capturing body 6 is an antibody.

The aggregation inhibitor 7 is a substance which can inhibit aggregation of the temperature-responsive macromolecule 1 when it exists near the temperature-responsive macromolecule 1. The aggregation inhibitor 7 is bonded to the site where it does not affect bonding of the second capturing body 6 to the target substance.

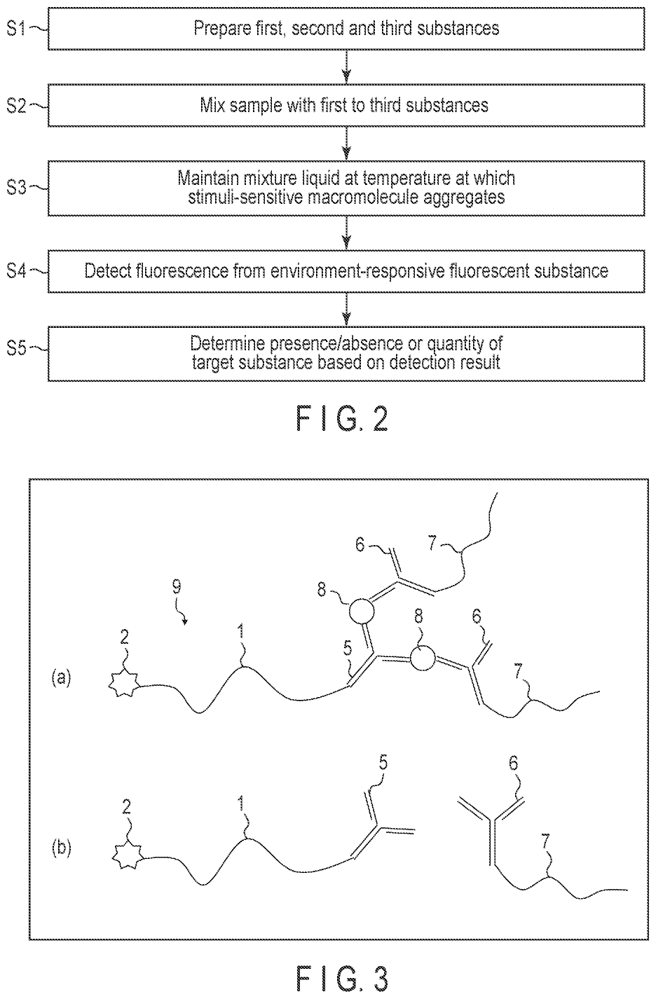

FIG. 2 briefly shows a flow of an example of the analytical method of the embodiment. The analytical method includes the following steps:

(S1) preparing the first substance, the second substance, and the third substance;

(S2) mixing the sample with the first substance, the second substance, and the third substance;

(S3) maintaining a mixture obtained by the step (S2) to a temperature at which a stimuli-sensitive macromolecule aggregates;

(S4) detecting fluorescence from the environment-responsive fluorescent substance; and

(S5) determining the presence/absence or quantity of a target substance in the sample based on a result of the detecting.

Hereafter, the principle of detection or quantification of a target substance by executing the above-provided steps will be described in detail.

Here, an example will be provided, in which the stimuli-sensitive macromolecule is the temperature-responsive macromolecule 1 and the environment-responsive fluorescent substance is the polarity-responsive fluorescent substance 2.

First, in step (S1), the first to third substances are prepared. Next, in step (S2), a sample and the first to third substances are mixed together. FIG. 3 shows a condition of each of the components in the mixture when mixing the sample and the first to third substances in a case where the target substance 8 exists in the sample.

By mixture (step (S2)), a complex 9 containing the target substance 8 can be formed. The complex 9 comprises, for example, the polarity-responsive fluorescent substance 2, the temperature-responsive macromolecule 1, the first capturing body 5, the target substance 8, the second capturing body 6, and the aggregation inhibitor 7, bonded to each other (see FIG. 3, part (a)).

In the case where the first capturing body 5 and the second capturing body 6 are substances which have a plurality of target substance-bonding sites as in this example, a further target substance 8 and a first capturing body 5 or a second capturing body 6 may bond to each of the target substance-binding sites.

On the other hand, when a sufficient amount of the target substance 8 is not present in the sample, there also exist the first to third substances which do not generate the complex 9 in the mixture (see FIG. 3, part (b)). In that case, the first substance and the second substance can bond each other. However, the third substance does not bind to them and remains separated.

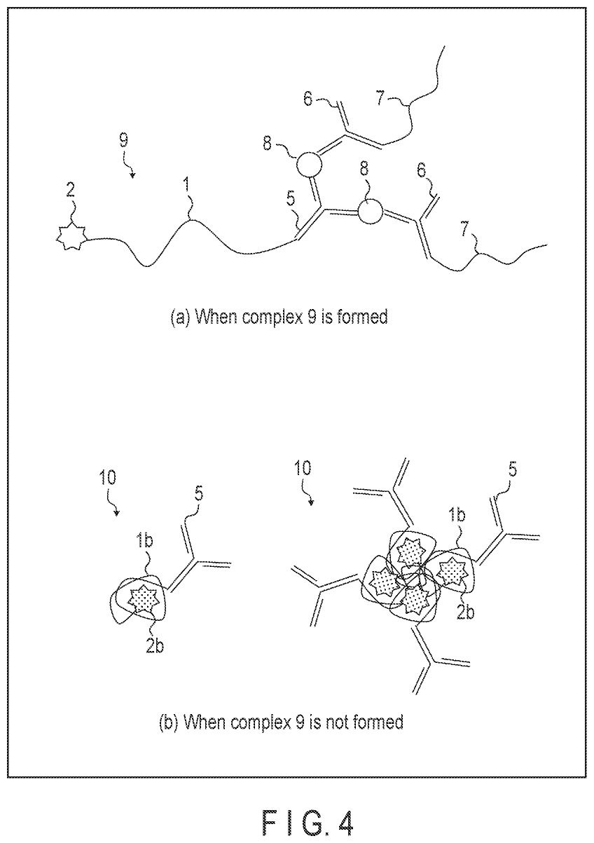

In step (S3), the mixture obtained by the mixing (Step (S2)) is maintained at a temperature at which the temperature-responsive macromolecule 1 aggregates. FIG. 4 shows condition of each component at that time. In the case where the first to third substances form the complex 9 (see FIG. 4, part (a)), the aggregation inhibitor 7 exists near a temperature-responsive macromolecule 1a. Therefore, the temperature-responsive macromolecule 1a is maintained in a hydrophilic state, thus inhibiting the aggregation. Therefore, the state that the surrounding of the polarity-responsive fluorescent substance 2a is maintained in hydrophilic, and the wavelength of the fluorescence does not change.

On the other hand, in the first and second substances which do not form the complex 9 (see FIG. 4, part (b)), the aggregation inhibitor 7 is not present near the temperature-responsive macromolecule 1b. Therefore, the temperature-responsive macromolecule 1b becomes hydrophobic to aggregate (the first substance and the second substance in which the temperature-responsive macromolecule 1b aggregates will be referred to as an “aggregate 10” hereinafter). As shown in FIG. 4 part (b), the aggregate 10 is formed as one temperature-responsive macromolecule 1b aggregates within the molecule, or as a plurality of temperature-responsive macromolecules 1b aggregate together.

In the aggregated temperature-responsive macromolecule 1b, a polarity-responsive fluorescent substance 2b is taken into the hydrophobic inside. That is, the polarity-responsive fluorescent substance 2b is present under a hydrophobic condition. Thus, the wavelength of fluorescence emitted by the polarity-responsive fluorescent substance 2b changes.

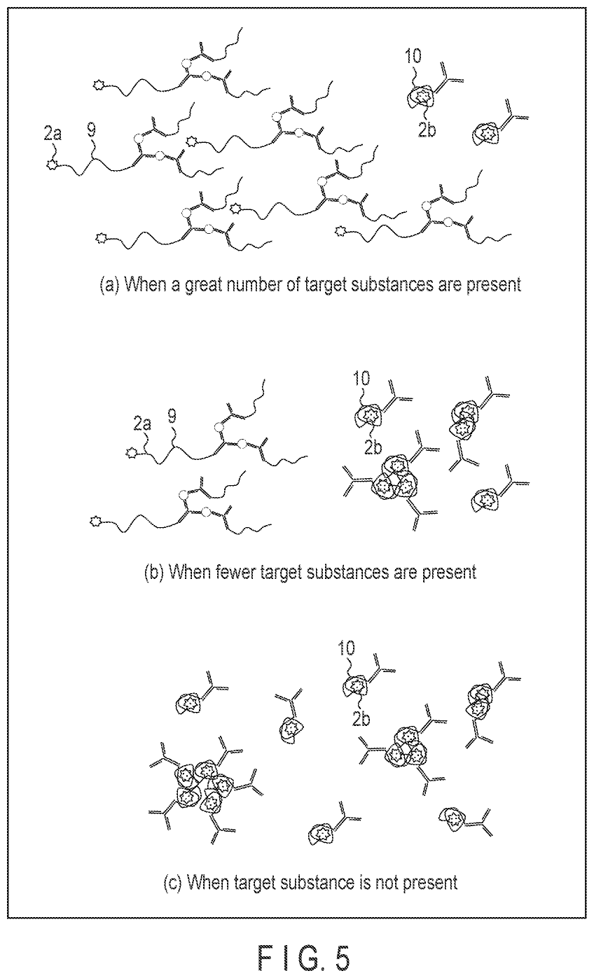

FIG. 5 shows the conditions of the first to third substances in samples containing different quantities of target substances. For example, when a number of target substances are present as shown in FIG. 5, part (a), more complexes 9 are formed. As a result, the number of polarity-responsive fluorescent substances 2a whose wavelength of fluorescence does not vary is greater than the number of polarity-responsive fluorescent substances 2b whose wavelength of fluorescence varied.

When there are fewer target substances as shown in FIG. 5, part (b), the number of polarity-responsive fluorescent substances 2a is less than the number of polarity-responsive fluorescent substances 2b.

When there is no target substance as shown in FIG. 5, part (c), the polarity-responsive fluorescent substance 2a is may not present, but only the polarity-responsive fluorescent substance 2b may present.

Next, in step (S4), fluorescence from the polarity-responsive fluorescent substance 2 is detected. The detection of fluorescence is carried out by, for example, the following manner. That is, the mixture is irradiated with excitation light of polarity-responsive fluorescent substances 2b whose wavelength of fluorescence varied, and fluorescence thus formed from the mixture is detected.

In that case, when a number of target substances are present, for example, as shown in FIG. 5, part (a), the intensity of fluorescence detected is low. When there are a fewer or no target substances as shown in FIG. 5, part (b) or (c), the intensity of fluorescence detected is higher than that of the case of FIG. 5, part (a). That is, as the number of target substances is more, the fluorescence detected becomes weak.

The detection of fluorescence may be carried out by irradiation of the excitation light of the polarity-responsive fluorescent substances 2b as described above, or by irradiation of excitation light of the polarity-responsive fluorescent substances 2a whose wavelength did not vary. In that case, the result of the intensity of fluorescence obtained is reversed. Or, excitation light of both the polarity-responsive fluorescent substances 2a and the polarity-responsive fluorescent substances 2b may be irradiated, and the intensity of fluorescence may be measured for both.

The detection of intensity of fluorescence may be carried out, for example, along with time. The term “along with time” is defined here that it may be carried out at a plurality of times with intervals or may be continuously carried out.

Next, in step (S5), the presence/absence or quantity of the target substance 8 in the sample is determined based on the result of the detection. For example, when the excitation light of the polarity-responsive fluorescent substances 2b whose wavelength of fluorescence varied is irradiated and fluorescence is not detected, it may be determine that the target substance is present. Or when the intensity of fluorescence is lower than a predetermined threshold, it may be determined that the target substance is present, and when higher than the threshold, it may be determined that the target substance is not present.

The threshold is predetermined by, for example, measuring the intensity fluorescence of a standard sample whose concentration of the target substance is already known. Alternatively, a calibration curve may be prepared by measurement of the intensity of fluorescence of a standard sample such as the above, and the quantity of the target substance of a sample to be analyzed may be determined according to the calibration curve.

Or, a calibration curve which indicates the relationship between the rise time of fluorescence and a target substance may be prepared, and the quantity of the target substance may be determined from the rise time of fluorescence.

According to the analytical method described above, the detection step is carried out by measuring the intensity of fluorescence from polarity-responsive fluorescent substances. With this structure, a target substance can be detected more precisely and in wider range as compared to the conventional techniques. With this method, the detection and quantification of a target substance can be carried out, for example, with precision of 100 to 1000 times or even higher as compared to the conventional procedure which uses temperature-responsive macromolecules. Moreover, the detection and quantification can be carried out even with higher precision as compared to those of the ELISA method and the CLEIA method.

In addition, according to this method, since fluorescence is utilized as an index, the result is hard to be affected by the influence by contaminants. For this reason, unlike the conventional techniques, it is not necessary to separate the mixture which contains a sample and a reagent, or to wash. Thus, the analytical method of this embodiment only requires to add the first to third substances in the sample and to control the temperature of the mixture. In this manner, contamination can be prevented and high-precision detection or quantification can be carried out far more simply than the conventional method. As described, since the procedure is easy, the analytical method of this embodiment can be carried out also using an apparatus provided for general analytical methods.

Samples used for the above-described analytical method are objects to be analyzed, which can contain target substances therein. An example of the samples is a liquid. The samples may be, for example, a biological material, a material originated from environment, a material originated from a food or drink, a material of industrial origin, an artificially produced formulation or a combination of any of those.

The target substance is, for example, a nucleic acid, a protein, an endocrine, a cell, a hemocyte, a virus, a microbe, an organic compound, an inorganic compound, or a low-molecular compound.

The temperature-responsive macromolecule 1 should preferably be, for example, a substance which is hydrophilic in a range of 0° C. to 30° C. and becomes hydrophobic to aggregate at 32° C. or higher. Usable examples of the temperature-responsive macromolecule 1 are polymers having a lower critical solution temperature, an1ggd polymers having a higher limit critical solution temperature.

Examples of the polymers which has a lower limit critical solution temperature are: polymers consists of N-substituted (metha)acrylamide derivatives such as N-n-propylacrylamide, N-isopropylacrylamide, N-ethylacrylamide, N,N-dimethylacrylamide, N-acryloylpyrrolidine, N-acryloylpiperidine, N-acryloylmorpholine, N-n-propylmethacrylamide, N-isopropylmethacrylamide, N-ethylmethacrylamide, N,N-dimethylmethacrylamide, N-methacryloylpyrrolidine, N-methacryloyl piperidine and N-methacryloyl morpholine; polyoxyethylenealkylamine derivatives such as hydroxypropylcellulose, partially acetylated polyvinyl alcohol, polyvinyl methyl ether, (polyoxyethylene-polyoxypropylene) block co-polymer and polyoxyethylenelaurylamine; a polyoxyethylene sorbitan ester derivative such as polyoxyethylene sorbitan laurate; (polyoxyethylene alkylphenylether) (metha)acrylates such as (polyoxyethylenenonylphenyl ether)acrylate and (polyoxyethyleneoctylphenyl ether) methacrylate; polyoxyethylene(metha)acrylic ester derivatives such as (polyoxyethylenealkyl ether)(metha) acrylates, for example, (polyoxyethylenelauryl ether) acrylate and (polyoxyethyleneoleyl ether)methacrylate. Further, copolymers of these, and polymers of at least two sorts of monomers of these can be used as well. Moreover, copolymers of N-isopropyl acrylamide and N-t-butyl acrylamide can be used as well. In the case of using a polymer containing a (metha)acrylamide derivative, some other copolymerizable monomers may be copolymerized with this polymer in a range which includes the lower limit maximum critical solution temperature.

An example of the polymer which has a lower limit critical solution temperature is a polymer formed of at least one sort of monomers selected from the group consisting of acloylglycinamide, acloylnipecotamide, acryloyl asparagine amide, acryloyl glutamic amide and the like, or, a copolymer which consists of at least two sorts of these monomers. With these polymers, some other copolymerizable monomers may be copolymerized in a range which includes the higher limit maximum critical solution temperature, examples of such are acrylamide, acetylacrylamide, biotinolacrylate, N-biotinyl-N′-methacloyltrimethylene amide, acloylsarcosine amide, methacrylsarcosine amide and acloylmethyl uracil.

The polarity-responsive fluorescent substance 2 is a substance whose wavelength of fluorescence changes, for example, at least about 400 nm to 700 nm, when the surroundings becomes hydrophobic from hydrophilic. As the polarity-responsive fluorescent substance 2, for example, POLARIC (registered trademark) or the like can be used.

The polarity-responsive fluorescent substance 2 can be bonded to the temperature-responsive macromolecule 1 using a method of a covalent bonding utilizing, for example, a carboxyl group or thiol group. For example, the polarity-responsive fluorescent substance 2 can be bonded to the temperature-responsive macromolecule 1 by combining the polarity-responsive fluorescent substance 2 with a polymerizing functional group such as a methacryl group or acryl group into an addition polymerizing monomer, and then copolymerizing with other monomers. Or it can carry out by, while polymerizing the polymers, copolymerizing a monomer which has a functional group such as carboxylic acid, an amino group, or an epoxy group, with other monomers, and covalently bonding via this functional group in accordance with a conventionally known procedure in this technical field.

Usable examples of the first capturing body 5 are an antibody, an antigen-binding fragment (for example, Fab, F(ab′)2, F(ab′), Fv, scFv or the like), a naturally derived nucleic acid, an artificial nucleic acid, an aptamer, a peptide aptamer, oligopeptide, enzyme and coenzyme.

In another embodiment, the first capturing body 5 and the other end 4 of the temperature-responsive macromolecule 1 may be bonded together in advance to prepare the first substance and the second substance as one body in step (S1). The bonding may be direct or indirect bonding. As will be described later in detail, both may be constituted to be bonded together via biotin and streptavidin.

Usable examples of the second capturing body 6 are an antibody, an antigen-binding fragment (such as Fab, F(ab′)2, F(ab′), Fv, scFv or the like), a naturally derived nucleic acid, an artificial nucleic acid, aptamer, a peptide aptamer, oligopeptide, enzyme and coenzyme.

The aggregation inhibitor 7 is a substance which inhibits aggregation of the temperature-responsive macromolecule 1, for example, when it approaches the temperature-responsive macromolecule 1 in term of distance. A usable example of the aggregation inhibitor 7 is a water-soluble macromolecule. Usable examples of the water-soluble macromolecule are natural polymers (such as polysaccharides of vegetable origin, water-soluble macromolecules originated from microorganisms, water-soluble macromolecules originated from animals), semisynthetic polymers (cellulose-based macromolecules, starch-based macromolecules and alginic acid macromolecules) and synthetic polymers (vinyl-based macromolecules).

It is preferable that the aggregation inhibitor 7 should be bonded to a site where it does not affect the binding to the target substance of the second capturing body 6. The aggregation inhibitor 7 can be bonded to the second capturing body 6 using any of the conventionally known procedures.

The first to third substances may be prepared in states of being contained in appropriate solvents, respectively. Examples of the appropriate solvents are aqueous solutions such as water and buffer solution.

For example, when a stimuli-sensitive macromolecule other than a temperature-responsive macromolecule is used in the analytical method of the embodiment, step (S3) can be carried out by maintaining the mixture under a specific condition by which the stimuli-sensitive macromolecule employed can be aggregated, that is, for example, conditions of a specific pH, light, and salt concentration.

In a further embodiment, the step (S2), i.e., the step of mixing the sample with the first to third substances may be carried out in the following two steps: (S2-1) the second substance and the third substance are mixed with the sample to bond the first capturing body and the second capturing body to the target substance; and (S2-2), subsequently, the first substance is added to the sample to bond the first capturing body to the other end of the temperature-responsive macromolecule, thus forming a complex.

In this manner, by separating the addition of the second and third substances and the addition of the first substance, and carrying out one after another, a greater amount of the second and third substances can be bonded to the target substance before carrying out the step (S3). A preferable example of such a method will be described later.

The analytical method of the embodiment described above can be employed for the detection or quantification of substances in various fields such as in vitro diagnosis of disease, diagnosis of microbial infection, food evaluation, and a doping test, for example. The analytical method of the embodiment is useful especially for detecting a micro dose of target substance contained in a sample.

Analytical Method Using Auto Analyzer

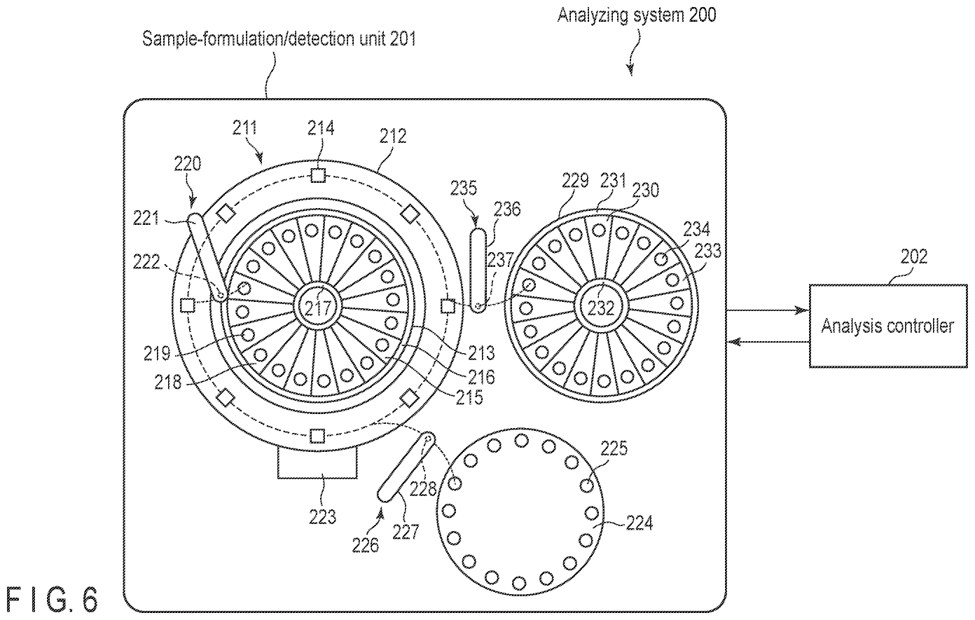

The analytical method of the embodiment can be carried out using an auto analyzer, for example. The auto analyzer comprises an analyzing system which, for example, adds the first to third substances to a sample, irradiates excitation light of a polarity-responsive fluorescent substance to measure fluorescence, and generates the data about the fluorescence. Such an analyzing system will be described using FIG. 6.

FIG. 6 is a plan view showing an example of an analyzing system 200. The analyzing system 200 comprises, for example, a sample-preparation/detection unit 201 and an analysis controller 202.

The sample-preparation/detection unit 201 comprises a reactor 211. The reactor 211 comprises an annular reaction disk 212, and an annular block 213 arranged within the reaction disk 212 coaxially therewith while maintaining a predetermined gap therebetween.

The reaction disk 212 rotates intermittently, for example, in a counterclockwise direction by a drive member (not shown). On the reaction disk 212, a plurality of reaction containers 214 are embedded along a circumferential direction thereof. From now, the locational relationship of the reaction disks 212 and the other members will be described, on the assumption that the reaction disk 212 is a clock board, for example, as 3 o'clock, 6 o'clock, 9 o'clock, 12 o'clock, etc.

On an annular block 213, circular recesses 215 are provided, and an outer circumferential ring 216 and an inner circumferential ring 217 are formed with respect the recesses 215. An outer circumferential surface of the annular block 213 comprises, for example, a rack in which a plurality of teeth (not shown) are engraved, and rotates intermittently, for example, in the counterclockwise direction with a drive gear to engage with the teeth of the rack. In the recesses 215 of the annular block 213, a plurality of second reagent containers 218 are fixed respectively along the circumferential direction. Each of the second reagent containers 218 has a tapered shape with one broad end, which narrows down towards the other end in width. Each of the second reagent containers 218 is provided to abut on the outer circumferential ring 216 by one end thereof, and abut on the inner circumference ring 217 by the other end, and to comprise a second reagent outlet 219 on a side of the one end abutting on the outer circumferential ring 216. A portion of the annular block 213, which is on an inner side with respect to the outer circumferential ring 216 functions as a second reagent cooling box.

A second reagent dispenser 220 comprises an arm 221 coupled to one end of a shaft (not shown) extending perpendicularly, which is located at a 10 o'clock position of the clock board of the reaction disk 212. The arm 221 is configured to be rotatable in both directions by with the shaft. The arm 221 comprises a flow path (not shown) inside, and also a suction/discharge nozzle 222 provided in a lower surface of the end on a side opposite to the shaft, which is communicated to the flow path. The suction/discharge nozzle 222 is ascended and descended by the arm 221. Note that a dispenser pump unit (not shown) is attached to an inner side of the arm 221. In the second reagent dispenser 220 with such a structure, one of the reaction containers 214 and one of the second reagent outlets 219 of the second reagent containers 218 are located under a trace (indicated by dashed line in the figure) of the suction/discharge nozzle 222 while reciprocal rotation of the arm 221.

A stirring arm (not shown) comprises an ascendable/descendible and rotatable stirring bar on a lower surface thereof. The stirring bar is placed at any position of the clock board of the reaction disk 212. In the stirring arm with such a structure, when the stirring bar is located right above the reaction container 214 to be subjected to the direction, which is moved by the counterclockwise rotation of the reaction disk 212, the stirring bar is descended and inserted to the reaction container 214, and then rotated to stir the liquid in the container 214.

The detection unit 223 is provided in an outer edge portion located at a 6 o'clock position of the clock board of the reaction disk 212. The detection unit 223 comprises an irradiation member (not shown) for irradiating excitation light towards the reaction container 214 to be detected, and a detector (not shown) which detects fluorescence from the reaction container 214 to which the excitation light is irradiated from the irradiation member.

A sample disk 224 is provided adjacent to a location at approximately the 5 o'clock position of the clock board of the reaction disk 212 of the reactor 211, so as to oppose. In an outer circumferential edge portion of the sample disk 224, a plurality of sample containers 225 are arranged and fixed along the circumferential direction, to contain, for example, samples or standard samples.

A sample dispenser 226 comprises an arm 227, one end of which is coupled with a shaft (not shown) extending perpendicularly. The arm 227 has a structure rotatable in both directions with the axis. The arm 227 comprises a flow path (not shown) and is provided with a suction/discharge nozzle 228 communicated with the flow path flow path on a lower surface of an end on an opposite side to the shaft. The suction/discharge nozzle 228 can be ascended and descended by the arm 227. Note that a dispenser pump unit (not shown) is attached to the inner side of the shaft. In the sample dispenser 226 with such a structure, one of the reaction containers 214 and one of the reaction containers 215 are located under a trace (indicated by dashed line in the figure) of the suction/discharge nozzle 228 while reciprocal rotation of the arm 227.

A circular block 229 for the first reagent is provided adjacent to the 3 o'clock position of the clock board of the reaction disk 212, so as to oppose. On the annular block 229 for the first reagent, circular recesses 230 are provided, and an outer circumferential ring 231 and an inner circumferential ring 232 are formed by the recesses 230. An outer circumferential surface of the annular block 229 for the first reagent comprises, for example, a rack in which a plurality of teeth (not shown) are engraved, and rotates intermittently, for example, in the counterclockwise direction with a drive gear to engage with the teeth of the rack. In the recesses 230 of the annular block 229 for the first reagent, a plurality of first reagent containers 233 are fixed respectively along the circumferential direction. Each of the first reagent containers 233 has a tapered shape with one broad end, which narrows down towards the other end in width. Each of the first reagent containers 233 is provided to abut on the outer circumferential ring 231 by one end thereof, and abut on the inner circumference ring 232 by the other end, and to comprise a first reagent outlet 234 on a side of the one end abutting on the outer circumferential ring 231. A portion of the annular block 229 for the first reagent, which is on an inner side with respect to the outer circumferential ring 231 functions as a first reagent cooling box.

A first reagent dispenser 235 comprises an arm 336, one end of which is coupled with a shaft (not shown) extending perpendicularly. The arm 236 has a structure rotatable in both directions with the axis. The arm 236 comprises a flow path (not shown) and is provided with a suction/discharge nozzle 237 communicated with the flow path flow path on a lower surface of an end on an opposite side to the shaft. The suction/discharge nozzle 237 can be ascended and descended by the arm 221. Note that a dispenser pump unit (not shown) is attached to the inner side of the arm 236. In the first reagent dispenser 226 with such a structure, one of the reaction containers 214 and one of the first reagent containers 233 are located under a trace (indicated by dashed line in the figure) of the suction/discharge nozzle 237 while reciprocal rotation of the arm 236.

The analysis controller 202 controls the intermittent rotation timing of the reaction disk 212, the annular block 213, the sample disk 224, and the circular block 229 for first reagents, controls the driving timing of the second reagent dispenser 220, the sample dispenser 226, the first reagent dispenser 235 and the stirring bar of the stirring arm, and controls also the irradiation timing of excitation light from the irradiation member, and the detection timing of the detection unit 223, etc. Moreover, the analysis controller 202 controls the temperature of the reaction container 214, the sample container 225, the first reagent cooling box, and the second reagent cooling box.

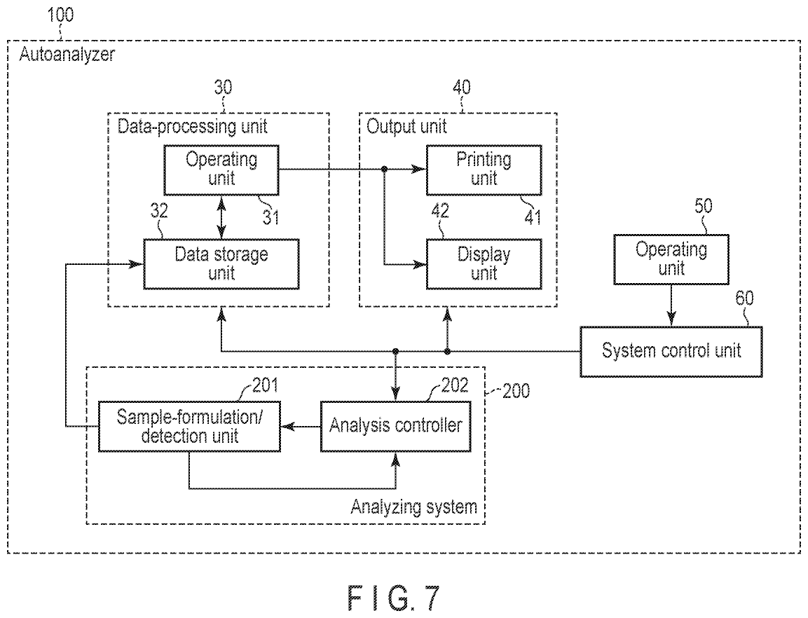

FIG. 7 is a block diagram showing an example of the auto analyzer 100.

The auto analyzer 100 comprises a data-processing unit 30 which receives the data on the fluorescence, created by the analyzing system 200, to process and generates data on the presence/absence or amount of the target substance (which will be referred to as “analytical data” hereinafter) and standard data. The data-processing unit 30 comprises an operating unit 31 and a storage unit 32. The operating unit 31 is related to analytical data and configured to generate standard data (for example, calibration data) which indicates the relationship between a fluorescent value and the concentration of a target substance. Moreover, the operating unit 31 is related to the sample to be analyzed, and configured to generate analytical data using the standard data. Furthermore, the storage unit 32 comprises a memory device and stores the standard data and analytical data generated by the operating unit 31.

The auto analyzer 100 comprises an output unit 40 which outputs data generated in the data-processing unit 30. The output unit 40 comprises a printing unit 41 which prints out the standard data or the analytical data, generated by the data-processing unit 30, and/or a display unit 42 which outputs and displays the data on a monitor or the like.

The auto analyzer 100 comprises an operating unit 50 which carries out an entry to set an analytic parameter required for analysis, an entry to start up the analyzing system 200, an entry to carry out a calibration and the like. The operating unit 50 comprises input devices such as a keyboard, a mouse, a button and a touch panel.

The auto analyzer 100 comprises an analysis controller 202 contained in the analyzing system 200, and a system control unit 60 which controls the data-processing unit 30 and the output unit 40. The system control unit 60 comprises a CPU and a storage circuit. The memory circuit stores the data entered from the operating unit 50, the program, the data regarding the fluorescence, the analytical data, the standard data and the like. The CPU controls the entire system by controlling the analysis controller 202, the data-processing unit 30 and the output unit 40 according to the input data and/or the program.

The analytical method to be carried out using the auto analyzer 100 described above will now be described.

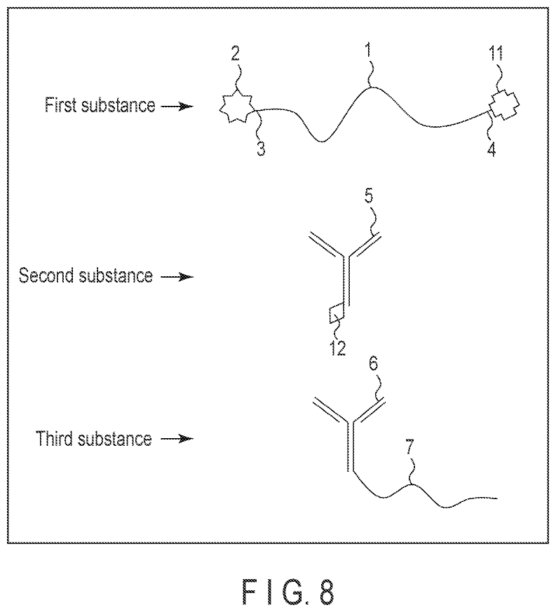

In this example, the first substance and the second substance are prepared as separate materials. FIG. 8 is a schematic diagram showing an example of the first to third substances used for this analytical method. In this example, the other end 4 of the temperature-responsive macromolecule 1 and the first capturing body 5 contain further components to bond them together. It suffices if the further components are two substances to bond to each other. It is preferable that these substances should be those having such a molecular weight that does not block the functions of the components of the first to third substances. Moreover, it is preferable that these two substances should be of an affinity higher than the affinity between the first and second capturing body and the target substance. Usable examples of these substances are biotin and streptavidin, protein A, protein G, melon gel and nucleic acid. In the example shown in FIG. 8, streptavidin 11 is bonded to the other end 4 of the temperature-responsive macromolecule 1 of the first substance, and biotin 12 is bonded to the first capturing body 5 of the second substance. For the other structures, similar structures described above can be employed.

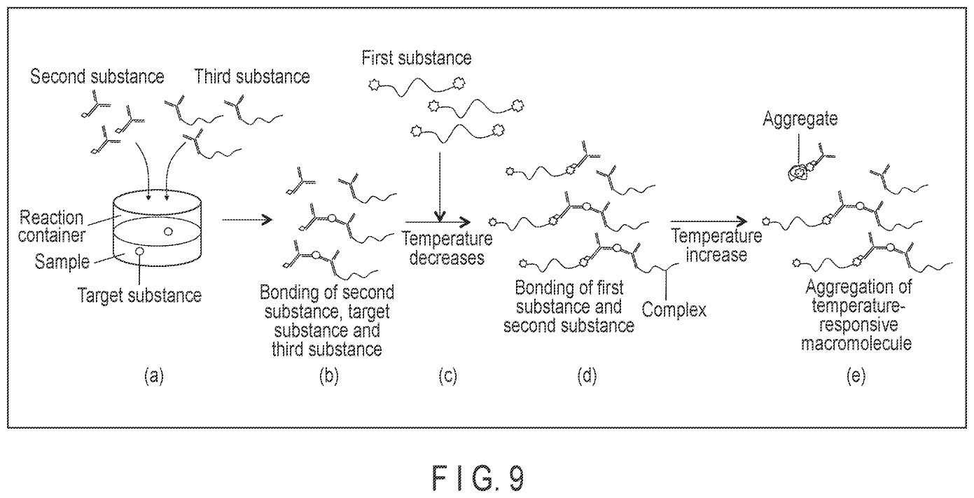

The process of the analytical method will now be described with reference to the analyzing system 200 shown in FIG. 6 and the schematic diagram of FIG. 9, which shows the behavior of the first to third substances.

First, different samples are accommodated, respectively, in the sample containers 225 of the auto analyzer 100. Then, the second reagent (that is, the first substance) of the same kind is accommodated in each of the second reagent containers 218, and the first reagent (that is, the second and third substances) of the same kind is accommodated in each of the first reagent containers 233. Each of the sample containers 225 is controlled by the analysis controller 202 to be maintained at 2° C. to 20° C. The second reagent container 218 and the first reagent container 233 are maintained by the second reagent cooling box and the first reagent cooling box, respectively, at 2° C. to 20° C.

Subsequently, the arm 227 of the sample dispenser 226 is rotated towards the sample disk 224, so that the suction and outlet nozzle 228 is moved to be located right above the sample container 225 in which the sample to be detected is accommodated, and then the tip of the suction/discharge nozzle 228 is descended to the sample in the sample container 225. Subsequently, the suction/discharge nozzle 228 suctions the sample accommodated in the sample container 225. Then, the suction/discharge nozzle 228 is ascended, and the arm 227 is rotated towards the reaction disk 212, to locate the suction/discharge nozzle 228 right above one reaction container 214 on the reaction disk 212. Thereafter, the tip of the suction/discharge nozzle 228 is descended into the reaction container 214. Then, the sample in the suction/discharge nozzle 228 is discharged into the reaction container 214 to inject the sample into the reaction container 214. Subsequently, the suction/discharge nozzle 228 is ascended, and the arm 227 is rotated to return it to the original position.

The reaction disk 212 is rotated in the counterclockwise direction to move the reaction container 214 containing the sample to a location corresponding to the 3 o'clock position of the clock board, which opposes the circular block 229 for the first reagent. Here, with regard to the first reagent dispenser 235, as in the case of the injection of the sample, the arm 236 is operated to rotate towards the circular block 229 for the first reagent. Thus, the first reagent is injected to the reaction container 214 from the first reagent container 233 to add the second substance and the third substance to the sample (see FIG. 9, part (a)).

Thereafter, the reaction disk 212 is rotated in the counterclockwise direction so as to locate the reaction container 214 directly under the stirring bar of the stirring arm (not shown). Then, the stirring bar is descended to the mixture in the reaction container 214 and rotated, thereby stirring the mixture. At this time, as shown in FIG. 9, part (b), the second substance, the target substance in the sample and the third substance are bonded together.

Next, the reaction disk 212 is rotated in the counterclockwise direction to move the reaction container 214 to the 9 o'clock position of the clock board. Here, with respect to the second reagent dispenser 220, as in the case of the injection of the sample, the arm 221 is operated to rotate towards the annular block 213. Thus, the second reagent is injected to the reaction container 214 from the second reagent container 218, to add it into the mixture in the reaction container 214. Due to addition of the second reagent (the first substance) (FIG. 9, part (c)), the temperature of the mixture contained in the reaction container 214 decreases. Moreover, as shown in FIG. 9, part (d), the first substance and the second substance are bonded together via the streptavidin 11 and the biotin 12, to form a complex. Here, since the reaction container 214 is controlled in advance to be maintained at 30° C. to 40° C., the mixture contained in the reaction container 214 then increases automatically to that temperature in about 1 minute to 30 minutes, for example. Thereby, the temperature-responsive macromolecule of the first substance which does not form the complex aggregates, to form an aggregate (FIG. 9, part (e)).

The time period from the step (a) of addition of the second and third substances to the sample, to the increase of the temperature, after the step (d), is about 1 minute to about 30 minutes, for example. This time period is sufficient for the first capturing body and the second capturing body to be bonded to the target substance. Meanwhile, the time period from the step (c) of addition of the first substance, to the increase of the temperature, after the step (d), is about 1 minute to about 30 minutes, for example. This time period is shorter than the time period immediately before the steps of (a) to (d), but this time period is sufficient for bonding, because the affinities of the streptavidin 11 and the biotin 12 are higher. With such a method, it is possible to prevent the temperature-responsive macromolecule 1 from aggregating, which may be caused by rising of temperature before bonding the first and second substances to the target substance. More specifically, in the case where bonding the first substance and the second substance together in advance, aggregation may occur before the second substance bonds to the target substance, because the time period from the addition of this bonded material to the rising of temperature is about 1 minute to about 30 minutes. On the other hand, according to the method, the addition of the second substance and the addition of the first substance are separated and the second substance is added first to allow a sufficient time for the second substance to bond to the target substance. In this manner, even if the time period until recovering of the temperature from the addition of the first substance is short, it is still possible to prevent the temperature-responsive macromolecule 1 from aggregating before the second substance bonds to the target substance.

After the temperature of the mixture in the reaction container 214 increased, the reaction disk 212 is rotated in the counterclockwise direction to place the reaction container 214 to oppose the detection unit 223 at the 6 o'clock position of the clock board. Then, the excitation light to excite the polarity-responsive fluorescent substance under hydrophobic conditions is irradiated from the irradiation member (not shown) of the detection unit 223 onto the mixture in the reaction container 214. Then, the fluorescence produced from the mixture in the reaction container 214 is detected by the detector (not shown) of the detection unit 223.

The data on the fluorescence, obtained by detection is sent to the data-processing unit 30 shown in FIG. 7, and the data (analytical data) regarding the presence/absence or quantity of the target substance and standard data are generated. The analytical data and the standard data are output to the output unit 40. A part or all of the steps described above can be automatically carried out by programs written.

In the automatic analysis of the target substance in the sample by the auto analyzer described above, the sample in the sample container 225 of the sample disk 224 is injected into the reaction container 214, and then the sample disk 224 is rotated, for example in the counterclockwise direction to move the sample container 225 by one section. Then, the first reagent in the first reagent container 233 of the circular block 229 for first reagents is injected to the reaction container 214, and thereafter the disk is rotated, for example, in the counterclockwise direction to move the first reagent container 233 by one section. Similarly, after injecting the second reagent in the second reagent container 218 of the annular block 213 into the reaction container 214, the annular block 213 is rotated, for example, in the counterclockwise direction to move the second reagent container 218 by one section. With such operation, the following samples are prepared for the automatic analysis.

As described above, the analytical method of the embodiment can be carried out by the auto analyzer. According to the analytical method of the embodiment, even if it is carried out by such an auto analyzer, it is not necessary to separate the mixture or wash it, for example, each time a reagent is sequentially added from the step of addition of the sample to the detection of fluorescence. Thus, the target substance can be detected or quantified with one reaction container 214, and therefore it is possible to prevent contamination and to carry out detection and quantification more simply at high precision.

Analytical Method Using a Competitive Method

In the further embodiment, the analytical method can be carried out using the competitive method.

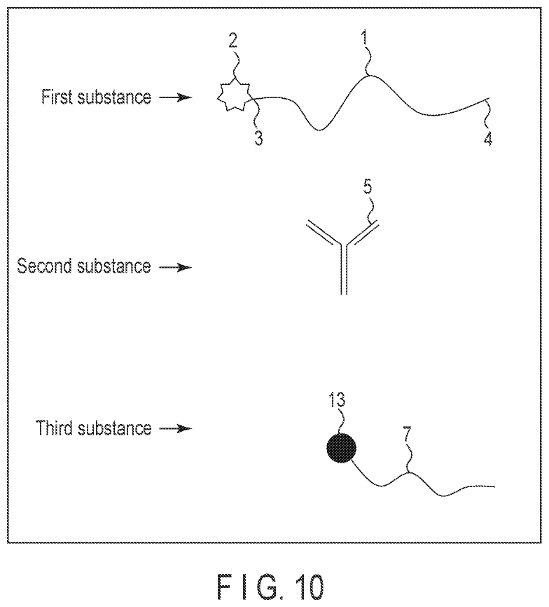

The first to third substances used for this method will be described with reference to FIG. 10. Here, the same first and second substances as any of those described above can be used here. The third substance contains a competitive substance 13 labeled with an aggregation inhibitor 7. The same aggregation inhibitor 7 as any of those described above can be used.

The competitive substance 13 is a substance which has affinity to the first capturing body and competes with a target substance in the binding to the first capturing body. The competitive substance comprises a site having a configuration similar to that of the binding site to the first capturing body of the target substance, for example. It is preferable that the affinity of the first capturing body 5 and the competitive substance 13, for example, be weaker than the affinity of the first capturing body 5 and the target substance.

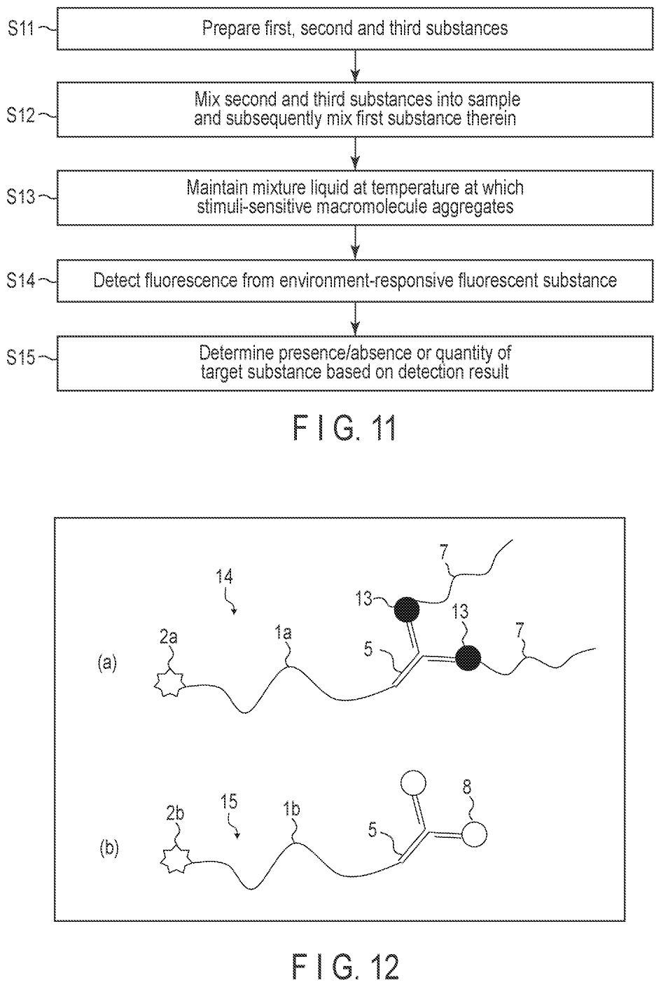

FIG. 11 shows a schematic flaw of an example of the analytical method using the competitive method. The analytical method comprises, for example, the following steps:

(S11) preparing the first substance, the second substance, and the third substance;

(S12) mixing the second substance and the third substance into the sample, and subsequently mixing the first substance therein;

(S13) maintaining the mixture obtained by the mixing (S12) at a temperature which a stimuli-sensitive macromolecule aggregates;

(S14) detecting fluorescence from an environment-responsive fluorescent substance; and

(S15) determining presence/absence or quantity of a target substance in the sample based on the result of the detecting.

The analytical method will be described below. Here, an example will be provided, in which the stimuli-sensitive macromolecule is the temperature-responsive macromolecule 1, and the environment-responsive fluorescent substance is the polarity-responsive fluorescent substance 2.

In step (S12), the first to third substances are mixed into a sample to form a first complex 14 as shown in FIG. 12, part (a). The first complex 14 comprises, for example, a polarity-responsive fluorescent substance 2a, a temperature-responsive macromolecule 1a, a first capturing body 5, a competitive substance 13, a second capturing body 6 and an aggregation inhibitor 7. In the case where the first capturing body 5 is a substance which has a plurality of target substance binding sites as in this example, a further competitive substance 13 and a further aggregation inhibitor 7 may be bonded to a plurality of target substance binding sites. Subsequently, when a target substance is presence in the sample, the bonding of the competitive substance 13 to the first capturing body 5 in the first complex 14 is substituted by the binding of the target substance 8, and thus a second complex 15 is formed (FIG. 12, part (b)). The second complex 15 comprises, for example, the polarity-responsive fluorescent substance 2b, the temperature-responsive macromolecule 1b, the first capturing body 5 and the target substance 8.

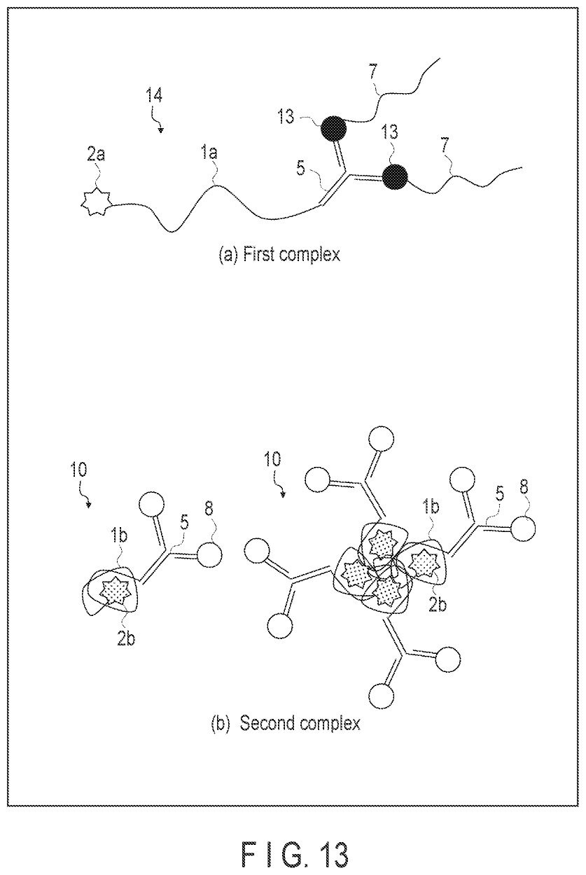

In step (S13), the temperature is maintained to which the temperature-responsive macromolecule aggregates. FIG. 13 shows the first complex 14 and the second complex 15 at that time. The temperature-responsive macromolecule 1a of the first complex 14 is present in the vicinity of the aggregation inhibitor 7, and thereby does not aggregate (FIG. 13, part (a)). Therefore, the wavelength of the fluorescence of the polarity-responsive fluorescent substance 2a does not vary. On the other hand, the temperature-responsive macromolecule 1b contained in the second complex 15 becomes hydrophobic to aggregate, thus forming an aggregate 10 (FIG. 13, part (b)). Therefore, the wavelength of the fluorescence of the polarity-responsive fluorescent substance 2b changes.

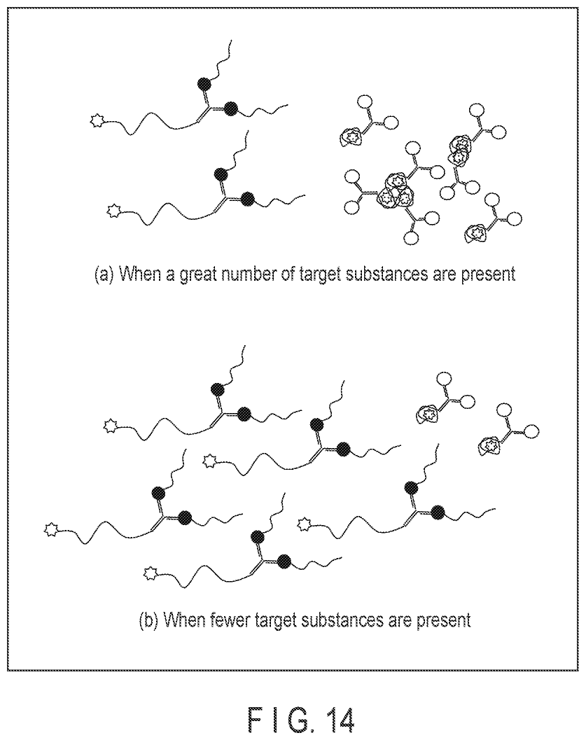

Under these circumstances, when a number of target substances are present as shown in FIG. 14, part (a), the substitution occurs more, and the number of the polarity-responsive fluorescent substances 2a whose wavelength of fluorescence does not vary is less than the number of the polarity-responsive fluorescent substance 2bs whose wavelength of fluorescence varied. When there are a less number of target substances as shown in FIG. 14, part (b), the number of the polarity-responsive fluorescent substances 2a is greater than the number of polarity-responsive fluorescent substance 2bs.

In step (S14), the fluorescence from the polarity-responsive fluorescent substance 2 is detected. The detection of fluorescence can be carried out by a method similar to that of the step (S4), for example. However, in the case where excitation light of the polarity-responsive fluorescent substance 2b whose wavelength of fluorescence varied is irradiated, the relationship between the existing amount of the target substance and the intensity of fluorescence obtained is contrary to that of the step (S4). That is, as there are a more number of target substances present, the intensity of fluorescence detected becomes higher.

For the detection of the fluorescence, the excitation light of the polarity-responsive fluorescent substance 2b may be irradiated as described above, or the excitation light of the polarity-responsive fluorescent substance 2a whose wavelength did not vary may be irradiated. In that case, with regard to the fluorescence intensity, a reverse result is obtained. Or, excitation light of both the polarity-responsive fluorescent substance 2a and the polarity-responsive fluorescent substance 2b may be irradiated to measure the fluorescence intensities of both.

In step (S15), the presence/absence or quantity of the target substance 8 in the sample is determined based on the result of the detection. For example, it may be determined that a target substance is present when the fluorescence is detected in the case where excitation light of the polarity-responsive fluorescent substance 2b whose wavelength of fluorescence varied is irradiated. Or, it may be determined that a target substance is present when the intensity of fluorescence is higher than a predetermined threshold, or that a target substance is present when the intensity is lower than the threshold.

The threshold is determined in advance, for example, by measuring the intensity of fluorescence with a standard sample whose concentration of the target substance is already known. Or, by measurement of the fluorescence intensity of such a standard sample, a calibration curve may be created to determine the quantity of the target substance of the sample to be analyzed according to the calibration curve. Or the quantity of a target substance may be determined based on the rise time of the fluorescence.

The analytical method using the competitive method described above can also be carried out using the auto analyzer 100. According to the analytical method using the competitive method, target substances having a lower molecular weight can be detected or quantified at higher precision.

Reagent Kit

According to a further embodiment, a reagent kit to be used for the analytical method of the embodiment is provided. The reagent kit of the embodiment contains, for example, the first substance, the second substance and the third substance of the embodiment. The first to third substances may be accommodated in separate containers, respectively, or the second and third substances may be accommodated together in the same container. Or, the other end 4 of the temperature-responsive macromolecule 1 of the first substance and the first capturing body of the second substance may be bonded together in advance and accommodated in one container.

The first to third substances may be contained, for example, in appropriate solvents described above.

While certain embodiments have been described, these embodiments have been presented by way of example only, and are not intended to limit the scope of the inventions. Indeed, the novel embodiments described herein may be embodied in a variety of other forms; furthermore, various omissions, substitutions, and changes in the form of the embodiments described herein may be made without departing from the spirit of the inventions. The accompanying claims and their equivalents are intended to cover such forms or modifications as would fall within the scope and spirit of the inventions.

1. An analytical method of detecting a target substance in a sample, comprising:
mixing a) a first substance containing a stimuli-sensitive macromolecule and an environment-responsive fluorescent substance bonded to one end of the stimuli-sensitive macromolecule, b) a second substance containing a first capturing body which bonds specifically to the target substance, and c) a third substance containing a second capturing body labeled with an aggregation inhibitor which inhibits aggregation of the stimuli-sensitive macromolecule, which bonds specifically to the target substance, with the sample;
maintaining the mixture under such a condition that the stimuli-sensitive macromolecule aggregates;
detecting fluorescence from the environment-responsive fluorescent substance; and
determining presence/absence or quantity of the target substance in the sample based on a result of the detecting.

2. The method of claim 1, comprising:
preparing a) a first substance containing a stimuli-sensitive macromolecule and an environment-responsive fluorescent substance bonded to one end of the stimuli-sensitive macromolecule, b) a second substance containing a first capturing body which bonds specifically to the target substance, and c) a third substance containing a second capturing body labeled with an aggregation inhibitor which inhibits aggregation of the stimuli-sensitive macromolecule, which bonds specifically to the target substance;
mixing the sample with the first substance, the second substance and the third substance, wherein when the target substance exists in the sample, the environment-responsive fluorescent substance, the stimuli-sensitive macromolecule, the first capturing body, the target substance, the second capturing body, and the aggregation inhibitor bond together to form a complex;
maintaining the mixture obtained by the mixing under such a condition that the stimuli-sensitive macromolecule aggregates, to aggregate the stimuli-sensitive macromolecule which does not form the complex and to allow the environment-responsive fluorescent substance bonded to the stimuli-sensitive macromolecule to exist under a hydrophobic condition;
detecting fluorescence from the environment-responsive fluorescent substance; and
determining presence/absence or quantity of the target substance in the sample based on a result of the detecting.

3. The method of claim 2, wherein
the mixing further comprises:
mixing the second substance and the third substance with the sample to bond the first capturing body and the second capturing body to the target substance; and
adding the first substance in the sample to bond the first capturing body and an other end of the stimuli-sensitive macromolecule to each other, thus forming a complex.

4. The method of claim 1, wherein
the first capturing body and the second capturing body are bonded to different binding sites of the target substance respectively.

5. An analytical method of detecting a target substance in a sample, comprising:
preparing a) a first substance containing a stimuli-sensitive macromolecule and an environment-responsive fluorescent substance bonded to one end of the stimuli-sensitive macromolecule, b) a second substance containing a first capturing body which bonds specifically to the target substance, and c) a third substance containing a competitive substance labeled with an aggregation inhibitor which inhibits aggregation of the stimuli-sensitive macromolecule, wherein the competitive substance is a substance having affinity to the first capturing body and competitive to the target substance in the binding to the first capturing body;
mixing the first to third substances with the sample;
maintaining the mixture under such a condition that the stimuli-sensitive macromolecule aggregates;
detecting fluorescence from the environment-responsive fluorescent substance; and
determining presence/absence or quantity of the target substance in the sample based on a result of the detecting.

6. The method of claim 5, comprising:
preparing a) a first substance containing a stimuli-sensitive macromolecule and an environment-responsive fluorescent substance bonded to one end of the stimuli-sensitive macromolecule, b) a second substance containing a first capturing body which bonds specifically to the target substance, and c) a third substance containing a competitive substance labeled with an aggregation inhibitor which inhibits aggregation of the stimuli-sensitive macromolecule, wherein the competitive substance is a substance having affinity to the first capturing body and competitive to the target substance in the binding to the first capturing body;
mixing the first to third substance with the sample to form a first complex comprising the environment-responsive fluorescent substance, the stimuli-sensitive macromolecule, the first capturing body, the competitive substance, and the aggregation inhibitor, and when the target substance exists in the sample, substituting the bonding of the first capturing body of the first complex to the competitive substance with the bonding to the target substance, to bond the stimuli-sensitive macromolecule, the first capturing body and the target substance, thus forming a second complex;
maintaining the mixture obtained by the mixing under such a condition that the stimuli-sensitive macromolecule aggregates, to aggregate the stimuli-sensitive macromolecule which does not form the first complex and to allow the environment-responsive fluorescent substance bonded to the stimuli-sensitive macromolecule to exist under a hydrophobic condition;
detecting fluorescence from the environment-responsive fluorescent substance; and
determining presence/absence or quantity of the target substance in the sample based on a result of the detecting.

7. The method of claim 5, wherein
a site of the first capturing body, which does not affect binding with the target substance, and the other end of the stimuli-sensitive macromolecule, to which the environment-responsive fluorescent substance is not bonded, are configured to bond to each other.

8. The method of claim 7, wherein
streptavidin is bonded to the other end of the stimuli-sensitive macromolecule of the first substance, and biotin is bonded to the first capturing body of the second substance.

9. The method of claim 5, wherein
the detecting of the fluorescence from the environment-responsive fluorescent substance comprises:
irradiating excitation light of the environment-responsive fluorescent substance under a hydrophobic condition to the mixture; and
detecting fluorescence from the mixture.

10. The method of claim 5, wherein
the stimuli-sensitive macromolecule is a temperature-responsive macromolecule.

11. The method of claim 5, wherein
the environment-responsive fluorescent substance is a polarity-responsive fluorescent substance.

12. A reagent kit for detecting a target substance in a sample, the kit comprising:
a first substance containing a stimuli-sensitive macromolecule and an environment-responsive fluorescent substance bonded to one end of the stimuli-sensitive macromolecule;
a second substance containing a first capturing body specifically bonded to the target substance; and
a third substance containing a second capturing body labeled with an aggregation inhibitor which inhibits aggregation of the stimuli-sensitive macromolecule, and specifically bonding to the target substance.

13. A reagent kit for detecting a target substance in a sample, the kit comprising:
a first substance containing a stimuli-sensitive macromolecule and an environment-responsive fluorescent substance bonded to one end of the stimuli-sensitive macromolecule;
a second substance containing a first capturing body specifically bonded to the target substance; and
a third substance containing a competitive substance labeled with an aggregation inhibitor which inhibits aggregation of the stimuli-sensitive macromolecule,
wherein
the competitive substance is a substance which has affinity to the first capturing body and competes with the target substance in binding to the first capturing body.

14. The reagent kit of claim 13, wherein
streptavidin is bonded to another end of the stimuli-sensitive macromolecule of the first substance, and biotin is bonded to the first capturing body of the second substance.

15. A device to be used the analytical method of claim 1, comprising:
an analytic system which mixes the sample with the first to third substances, irradiates excitation light of the environment-responsive fluorescent substance onto the mixture to measures fluorescence, and generates data regarding the fluorescence; and
a data-processing unit which generates data regarding presence/absence or quantity of the target substance from the data on the fluorescence.

16. The method of claim 1, wherein
a site of the first capturing body, which does not affect binding with the target substance, and the other end of the stimuli-sensitive macromolecule, to which the environment-responsive fluorescent substance is not bonded, are configured to bond to each other.

17. The method of claim 1, wherein
the detecting of the fluorescence from the environment-responsive fluorescent substance comprises:
irradiating excitation light of the environment-responsive fluorescent substance under a hydrophobic condition to the mixture; and
detecting fluorescence from the mixture.

18. The method of claim 1, wherein
the stimuli-sensitive macromolecule is a temperature-responsive macromolecule.

19. The method of claim 1, wherein
the environment-responsive fluorescent substance is a polarity-responsive fluorescent substance.

20. The reagent kit of claim 12, wherein
streptavidin is bonded to another end of the stimuli-sensitive macromolecule of the first substance, and biotin is bonded to the first capturing body of the second substance.
